(19) United States
(12) Patent Application Publication
(10) Pub. No.: US 2022/0026342 A1
(43) Pub. Date: Jan. 27, 2022

(54) PORTABLE MULTIFUNCTIONAL TESTER FOR POLLUTANT EROSION EFFECT AND UNIDIMENSIONAL CONSOLIDATION AND TEST METHOD THEREOF

(71) Applicants: Zexiang Wu, Wenzhou City (CN); Chuang Yu, Wenzhou City (CN); Xiaoqing Cai, Wenzhou City (CN); Dongmei Tu, Wenzhou City (CN)

(72) Inventors: Zexiang Wu, Wenzhou City (CN); Chuang Yu, Wenzhou City (CN); Xiaoqing Cai, Wenzhou City (CN); Dongmei Tu, Wenzhou City (CN)

(73) Assignee: Wenhou University

(21) Appl. No.: 16/939,085

(22) Filed: Jul. 27, 2020

(51) Int. Cl. G01N 17/00; G01N 33/24

(52) U.S. Cl. CPC G01N 17/002; G01N 2203/0048; G01N 33/24

(57) ABSTRACT

A portable multifunctional tester for pollutant erosion effect, including a base, the ejection head of jacking device is provided with a container. The upper end and lower end of the sample are provided with an upper porous stone and a lower porous stone respectively. A metal cover plate is disposed on the upper porous stone. An ejector rod with a circular tray is fixed to the support. The stress controlled one-dimensional load test can be performed by putting weights on the circular tray. A pressure gauge is disposed on the ejector rod. A displacement meter is disposed on the top cover plate. The container has a water inlet and a water outlet connected to its inner chamber. The water inlet, circulating pump, accumulator and water outlet are connected by conduits. In addition, a unidimensional consolidation test and washing test method thereof is provided.

### BACKGROUND OF INVENTION

### 1. Field of the Invention

The present invention relates generally to a portable multifunctional tester for pollutant erosion effect, and more particularly to a unidimensional consolidation and test method thereof.

### 2. Description of Related Art

At present, the stress on soil is complex, the coupling effect of pollutant erosion and temperature shall be considered in mechanical properties. However, the conventional consolidometer has not considered this effect. The conventional consolidometer generally has a base, a container is disposed on the base, and there is a sample box in the container. An upper porous stone and a lower porous stone are disposed on the upper end and lower end of sample respectively. The upper porous stone is provided with a metal cover plate. The metal cover plate is connected to a support, and the ejector rod is provided with a pressure gauge. The top cover plate is provided with a displacement meter. The ejector pin of displacement meter props the gland. However, this conventional consolidometer cannot test the effect of water flow and pollutants on erosion and washing of soil mass, the effect of pollutant erosion mid temperature has not been clarified, the calculation and design of the bearing capacity and settlement of soil mass will be influenced, severely obstructing the development of technical field of geotechnical engineering.

### SUMMARY OF THE INVENTION

The present invention provides a portable multifunctional tester for pollutant erosion effect. The tester can obtain more accurate consolidation test data and washing test data by simulating the flow velocity, temperature, and water composition of the original water body. Moreover, the present invention provides a unidimensional consolidation and test method thereof.

The purpose of the present invention is to overcome the shortcomings of the prior art and provide an output protection device for a charger. In order to solve the above technical problems, the present invention adopts the following first technical solution:

A portable multifunctional tester for pollutant erosion effect, comprising base, a support mounted on the base, a top cover plate disposed on a support, a jacking device disposed on the base, a strain controlled one-dimensional load test can be performed by using the jacking device; a container disposed on an ejection head of the jacking device; a sample box disposed in the container, the sample box can load samples; an ejector rod disposed on the support; a circular tray fixed to an upper end of the ejector rod; a gland disposed on a lower end of the ejector rod; the stress controlled one-dimensional load test can be performed by putting weights on the circular tray; a pressure gauge disposed on the ejector rod; the lower end of ejector rod being aligned with the upper porous stone; a displacement meter disposed on the top cover plate; the ejector pin of the displacement meter contacting the gland; the container having a water inlet and a water outlet connected to its inner chamber; wherein the water inlet, circulating pump, accumulator and water outlet are connected by conduits.

A unidimensional consolidation and washing test method thereof using the portable multifunctional tester for pollutant erosion effect in Claim 4, comprising the following steps:

the undisturbed sample I is taken in situ using the sample box, and the undisturbed water is taken from the river nearby the site;

the sample I is put in sample box to form sample, a lower porous stone 8 is disposed on the lower end of sample, an upper porous stone is disposed on the upper end of sample, filled with the undisturbed water for saturated immersion;

the circulating pump and/or heating unit is actuated to simulate the flow of water body and the original ambient temperature of water body, the water flow velocity is controlled by controlling the circulating pump to be coincident with the water velocity of the original water body; afterwards, the jacking device is actuated to make the ejector rod props the upper porous stone downwards for strain controlled consolidation test, or the jacking device is turned off, the weights are applied to the circular tray to perform the stress controlled consolidation test;

the jacking device, circulating pump and heating unit are turned off, and the outer clamping plate of right side plate is removed according to the primal environment of sample I, the buffer balls wrapped in a net bag are disposed on the outer side of inner baffle of rigid side plate, and the circulating pump and/or heating unit is actuated again to simulate the flow of water body and the original ambient temperature of water body for a period of time, the mechanical data after the soil mass is washed are tested again, and the morphology of the sample II after washing is filmed;

the data from Step C and Step D are combined for analysis.

Effects of the present invention:

1. The tester and test method thereof can simulate the flow velocity, temperature and washing mode of water body by circulating the undisturbed water in the original polluted water body, so that more accurate consolidation test data and washing test data of the undisturbed sample can be obtained in the tester.

2. The tester has the functions of consolidation test and washing test, favorable for keeping the water flow and temperature environment consistent in consolidation test and washing test, the accuracy of test data is further enhanced, and the equipment is simplified, it is convenient to take the equipment to the field for tests.

### BRIEF DESCRIPTION OF THE DRAWINGS

FIG. 1 is a structural representation of Embodiment 1 of the portable multifunctional tester for pollutant erosion effect of the present invention. The tester can perform the consolidation test considering temperature and pollutant erosion effect;

FIG. 2 is a structural representation of Embodiment 2 of the portable multifunctional tester for pollutant erosion effect of the present invention. The tester can perform the consolidation test considering temperature and pollutant erosion effect;

FIG. 3 is a schematic plan of the structure of bottom plate in FIG. 2;

FIG. 4 is a schematic plan of the structure of top plate in FIG. 2;

FIG. 5 is a section view of the structure at the junction of joint sealing strip and the end of side plate.

FIG. 6 is a structural representation of sample box;

FIG. 7 is an enlarged section view of the structure of clip on the ejector rod.

### DETAILED DESCRIPTION OF THE INVENTION

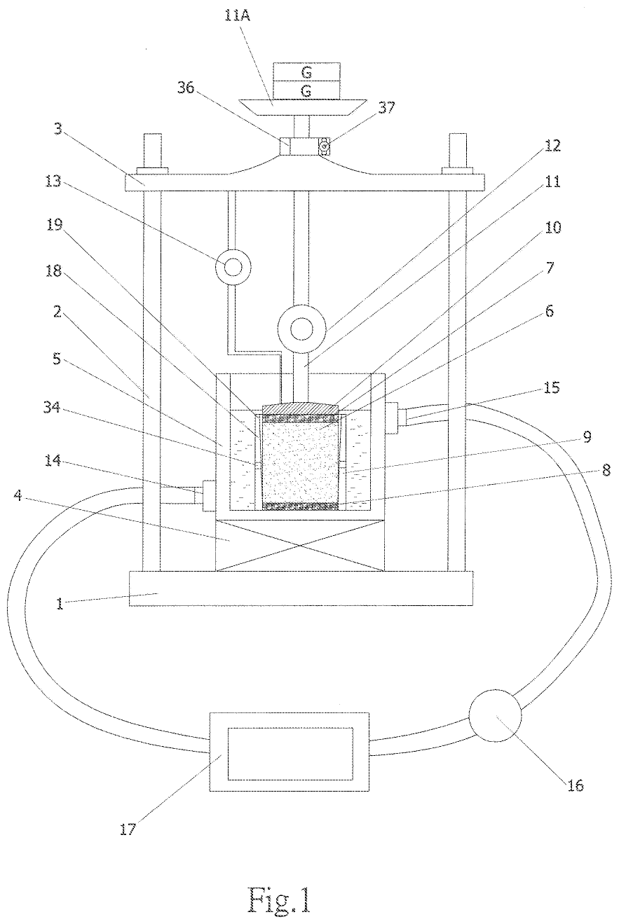

FIG. 1 shows the first embodiment of the portable multifunctional tester for pollutant erosion effect of the present invention, including a base 1, a support 2 is disposed on the base 1, a top cover plate 3 is disposed on the support 2. A jacking device 4 is disposed on the base 1, the strain controlled one-dimensional load test can be performed by using the jacking device 4. A container 5 is disposed on the ejection head of jacking device 4. A sample box 18 is disposed in the container. The sample box 18 can load a sample 6. The support 2 is provided with an ejector rod 11. A clip 36 is fixed to the upper end of top cover plate 3 (when strain controls load, the clip 36 is screwed down, the lower jacking device applies load, the clip 36 tightens ejector rod 11 to the top cover plate 3). A circular tray 11A is fixed to the upper end of ejector rod 11. A gland 10 is disposed on the lower end of ejector rod 11. The upper end and lower end of the sample box 18 are provided with an upper porous stone 7 and a lower porous stone 8 respectively. The gland 10 props the upper porous stone 7, the operator can put weights on the circular tray 11A to perform stress controlled one-dimensional load test (when stress controls load, the clip 36 is loosened and the jacking displacement is fixed, the upper weights apply load). A pressure gauge 12 is disposed on the ejector rod 11. The lower end of ejector rod 11 is aligned with the upper porous stone 7. A displacement meter 13 is disposed on the top cover plate 3. The ejector pin of displacement meter 13 contacts the gland 10. The container 5 has a water inlet water inlet 15 and a water outlet 14 connected to its inner chamber. The water inlet 15, circulating pump 16, accumulator 17 and water outlet 14 are connected by conduits. The casing of the accumulator 17 is wrapped in a heating unit. The heating unit can be a heating wire, the heating wire is provided with a power input control mechanism. The heating unit is wrapped in an insulation envelope. The external conductor is conductively connected to the heating wire through the insulation envelope. The heat is generated after the power is switched on. A temperature sensor is disposed in the accumulator 17. A display equipment for displaying the temperature data measured by the temperature sensor is disposed on the insulation envelope. The display equipment and heating wire are connected to the electroheat controller. The electroheat controller regulates temperature, favorable for setting different circulating water temperatures. The structure and principle of electroheat are the existing technology, which is not described anymore.

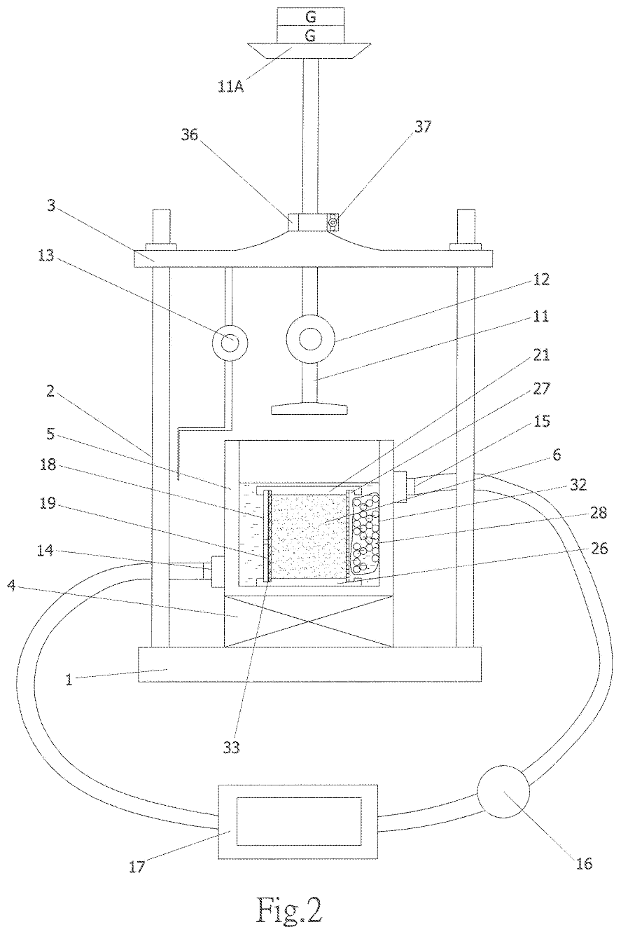

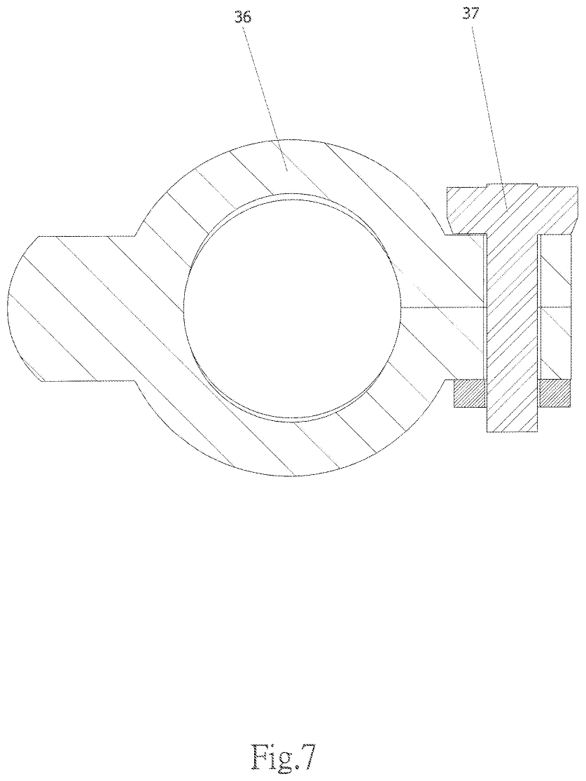

As shown in FIGS. 1, 2 and 7, in the aforethe embodiments, for convenient switchover of test modes, FIG. 7 shows that a clip 36 is fixed to the upper end of the top cover plate, the clip 36 is fitted over the ejector rod 11. There is an opening on one side of clip 36, the opening is adjusted by a bolt 37, the end of bolt 37 is provided with a regulating handle. As shown in FIGS. 1 and 2, in this embodiment, the clip 36 is fastened to the ejector rod 11, and when the dip 36 clamps the upper side of top cover plate 3, the ejector rod 11 is localized and suspended above container 5. The tests other than one-dimensional load test can be performed, e.g. the test for the effect of current scour on soil mass.

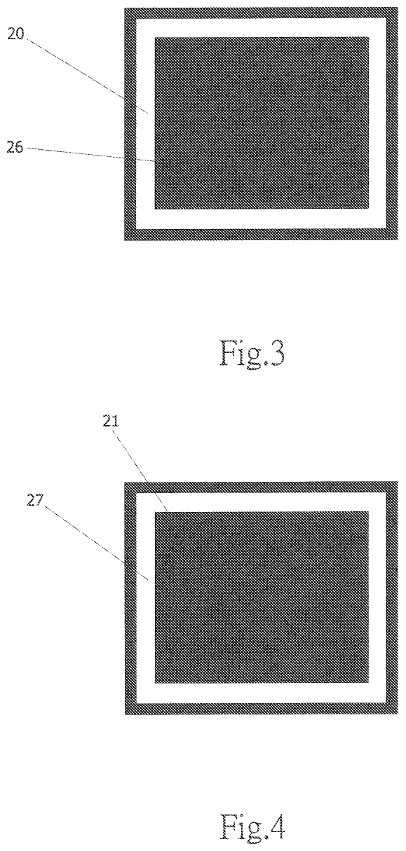

FIGS. 2-6 show the second embodiment of the portable multifunctional tester for pollutant erosion effect of the present invention, which is basically identical with the first embodiment, the difference is that the container 5 is provided with a sample box 18. The sample box 18 comprises a top plate 21, four side plates 19 (side plates are provided with a switchgear for controlling open/close of permeable circular holes) and a bottom plate 26. The bottom plate 26 is made of seepy material, the bottom plate 26 is generally made of a metal plate with pervious holes. The bottom plate 26 has a lower rectangular groove 20. The side plates 19 are inserted in the lower rectangular groove 20 to form side guard plates. The side plates include a left side plate 19A, a right side plate 19B, a front side plate 19C and a back side plate 19D. The upper end of the side plates 19 is provided with a top plate 21. The lower side face of top plate 21 has an upper rectangular groove 27 matching the lower rectangular groove 20. The side plates 19 are stuck in the upper rectangular groove 27 respectively. The left side plate 19A and right side plate 19B are provided with circular holes 34 for water-in or water-out. When the soil sample is put in the sample box 18, a partial side plate 19 can be removed, the partial side plate 19 is provided with multiple side plates 19 with circular holes 34 in different radii and quantities for replacement. The input/output water flow and washing position are regulated by using different side plates to perform the washing test.

As shown in FIG. 2, in the second embodiment, the portable multifunctional tester for pollutant erosion effect is provided with multiple buffer balls 28, the buffer balls 28 are filled on one side of sample 6 where the partial side plate 19 is removed. One side of the sample 6 filled with buffer balls 28 is provided with the water inlet 15, the water flow scours the buffer balls 28, the buffer balls 28 make sure that the water flow is loaded on the side of sample 6 in plan form.

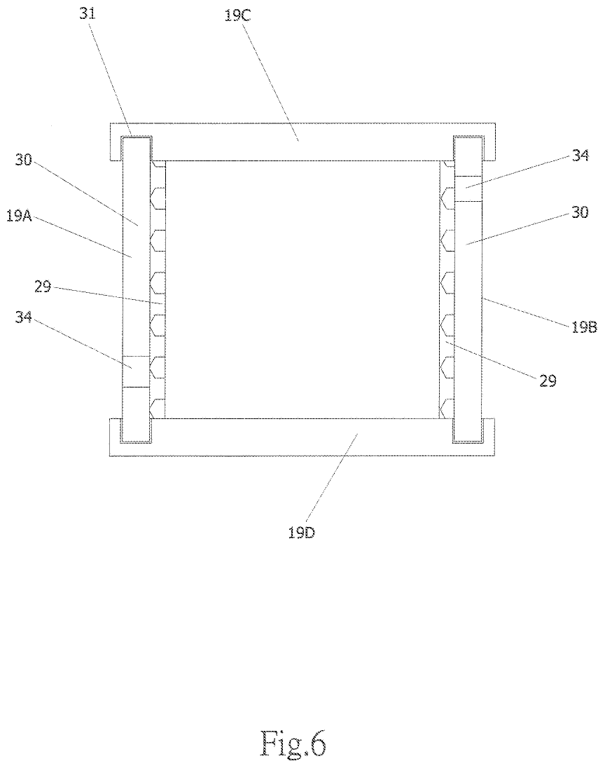

As shown in FIG. 6, in the second embodiment, for convenient washing test and maintaining the sample, in the aforethe embodiment, the left side plate 19A and right side plate 19B comprise an inner baffle 29 and an outer clamping plate 30 respectively. Multiple through holes are densely distributed over the inner baffle 29. The inner sides of both ends of the front side plate 19C and back side plate 19D are provided with a slideway 31 respectively. The outer clamping plate 30 is inserted in the slideway 31 from top to bottom for fixing. The inner baffle 29 is on the inner side of outer clamping plate 30. Alternatively, the inner baffle 29 and outer clamping plate 30 can be inserted in the slideway 31 from top to bottom for fixing. Generally, the end of outer clamping plate 30 is provided with a sealing strip. Multiple buffer balls 28 wrapped in a net bag 32 are disposed outside the right side plate 19B. A filter net bag 33 is disposed between the outer clamping plate 30 and inner baffle 29 of the left side plate 19A.

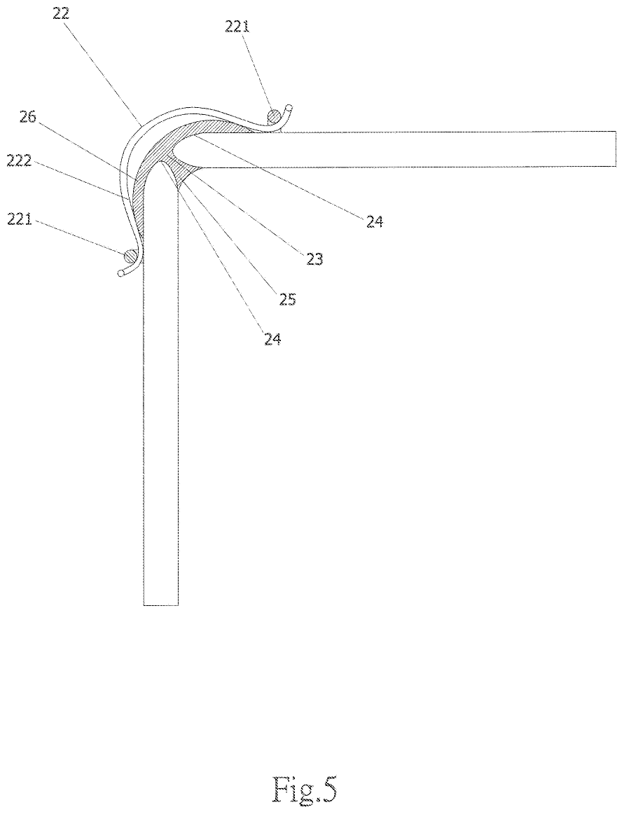

FIGS. 5 and 6 show the third embodiment of the portable multifunctional tester for pollutant erosion effect of the present invention, which is basically identical with the second embodiment the difference is the locking linkage structure and seal structure among the side plates 19, described below. For convenient assembly and disassembly of sample box and ensured sealing, the side plates 19 are fixed by locking mechanisms 22. A joint sealing strip 23 is disposed between side plates 19. The locking mechanism 22 comprises a hook 221 and a spring jig 222 fixed to the outer end of each side plate. The end of spring jig 22 is provided with a hooking piece for hooking the hook 221. The spring jig 222 hitches the hooks 221 of two side plates 19 to tighten the two side plates 19. The joint sealing strip 23 is made of sealing rubber. The end of side plate 19 is cambered. Both sides of joint sealing strip 23 are provided with a cambered slot 24 matching the end of side plate 19. There is an inner spacer plate 25 between two cambered slots 24. There is an outer side plate 26 on the outer side of joint sealing strip 23. The end of the side plate 19 is stuck in the cambered slot 24 and clamping the inner spacer plate 25. The outer side plate 26 of the joint sealing strip 23 adheres to the side plate 19 depending on the elasticity of rubber. Here the outer side plate 26 is a part of the joint sealing strip made of rubber material, so there is an inward clamping force on side plate 19. The aforethe structure is favorable for flexible removal of side plate 19, convenient for various washing tests. The portable multifunctional tester for pollutant erosion effect provided in this embodiment can be used for the consolidation test for pollutant effect, as well as for insitu washing test.

The present invention provides the unidimensional consolidation and washing test method thereof using the aforethe tester, comprising the following steps.
A. The undisturbed sample I is taken in situ using the sample box, and the undisturbed water is taken from the river nearby the site.
B. The sample I is put in sample box 18 to form sample 6, a lower porous stone 8 is disposed on line lower end of sample 6, an upper porous stone 7 is disposed on the upper end of sample 6, filled with the undisturbed water for saturated immersion.
C. The circulating pump 16 and/or heating unit is actuated to simulate the flow of water body and the original ambient temperature of water body, the water flow velocity is controlled by controlling the circulating pump 16 to be coincident with the water velocity of the original water body. Afterwards, the jacking device 4 is actuated to make the ejector rod 11 props the upper porous stone 7 downwards for strain controlled consolidation test, or the jacking device 4 is turned off, the weights are applied to the circular tray 11A to perform the stress controlled consolidation test.
D. The jacking device 4, circulating pump 16 and heating unit are turned off, and the outer clamping plate 30 of right side plate 19B is removed according to the primal environment of sample I, the buffer balls 28 wrapped in a net bag 32 are disposed on the outer side of inner baffle 29 of right side plate 19B, and the circulating pump 16 and/or heating unit is actuated again to simulate the flow of water body and the original ambient temperature of water body for a period of time, the mechanical data after the soil mass is washed are tested again, and the morphology of the sample II after washing is filmed.
E. The data from Step C and Step D are combined for analysis.

We claim:

1. A portable multifunctional tester for pollutant erosion effect, comprising:
a base, a support mounted on the base, a top cover plate disposed on a support, a jacking device disposed on the base, a strain controlled one-dimensional load test can be performed by using the jacking device; a container disposed on an ejection head of the jacking device; a sample box disposed in the container, the sample box can load samples; an ejector rod disposed on the support; a circular tray fixed to an upper end of the ejector rod; a gland disposed on a lower end of the ejector rod; the stress controlled one-dimensional load test can be performed by putting weights on the circular tray; a pressure gauge disposed on the ejector rod; the lower end of ejector rod being aligned with the upper porous stone; a displacement meter disposed on the top cover plate; the ejector pin of the displacement meter contacting the gland; the container having a water inlet and a water outlet connected to its inner chamber; wherein the water inlet, circulating pump, accumulator and water outlet are connected by conduits.

2. The portable multifunctional tester tor pollutant erosion effect defined in claim 1, wherein an upper end and a lower end of the sample box are provided with an upper porous stone and a lower porous stone respectively; the gland props the upper porous stone.

3. The portable multifunctional tester for pollutant erosion effect defined in claim 1, wherein the sample box is a square sample box; the sample box including a top plate, four side plates and a bottom plate; the bottom plate is made of permeable material; the bottom plate has a lower rectangular groove; the four side plates are inserted in the lower rectangular groove; the upper end of the side plates is provided with the top plate; a lower side face of the top plate is provided with an upper rectangular groove matching the lower rectangular groove; the side plates are stuck in the upper rectangular groove respectively; the side plates include a left side plate, a right side plate, a front side plate and a back side plate; the left side plate and right side plate are provided with circular holes for water-in or water-out; when the soil sample is put in the sample box, the partial side plate can be removed; the partial side plate is provided with multiple side plates with circular holes in different radii and quantities for replacement; the input/output water flow and washing position are regulated by using different side plates for washing test.

4. The portable multifunctional tester for pollutant erosion effect defined in claim 1, wherein the casing of the accumulator is wrapped in a heating unit, the heating unit is wrapped in an insulation envelope, there is a temperature sensor in the accumulator, there is a display equipment for displaying the temperature data measured by the temperature sensor on the insulation envelope.

5. The portable multifunctional tester for pollutant erosion effect defined in claim 3, wherein the portable multifunctional tester for pollutant erosion effect is provided with multiple buffer balls, the buffer balls are filled on one side of the sample where the partial side plate is removed, one side of the sample filled with buffer balls is provided with the water inlet, the water flow scours the buffer balls, the buffer balls make sure that the water flow is loaded on the side of sample in plan form; the side plates are fixed by locking mechanisms; a joint sealing strip is disposed between side plates.

6. The portable multi functional tester for pollutant erosion effect defined in claim 3, the left side plate and right side plate comprise an inner baffle and an outer clamping plate respectively; multiple through holes are distributed over the inner baffle; the inner sides of both ends of the front side plate and back side plate are provided with a slideway respectively; the outer clamping plate is inserted in the slide way from top to bottom for fixing; the inner baffle is on the inner side of outer clamping plate; multiple buffer balls wrapped in a net bag are disposed on the outer side of the right side plate: a filter net bag is disposed between the outer clamping plate and inner baffle of the left side plate.

7. The portable multifunctional tester for pollutant erosion effect defined in claim 3, wherein the locking mechanism includes a hook, and a spring jig fixed to the outer end of each side plate, the end of spring jig is provided with a hooking piece for hitching the hook; the spring jig hitches the hooks of two side plates to tighten two side plates.

8. The portable multifunctional tester for pollutant erosion effect defined in claim 7, wherein the joint sealing strip is made of sealing rubber, the end of side plate is cambered, both sides of joint sealing strip are provided with a cambered slot matching the end of side plate respectively; there is an inner spacer plate between two cambered slots; there is an outer side plate on the outer side of joint scaling strip; the end of the side plate is stuck in the cambered slot and clamping the inner spacer plate; the outer side plate adheres to the side plate depending on the elasticity of rubber.

9. The portable multifunctional tester for pollutant erosion effect defined in claim 1, wherein a clip is fixed to the upper end of the top cover plate, the clip is fitted over the ejector rod, one side of clip has an opening, the tension of opening is adjusted by a bolt, the bolt end is provided with a regulating handle.

10. A unidimensional consolidation and washing test method thereof using the portable multifunctional tester for pollutant erosion effect in claim 4, comprising the following steps:
A. the undisturbed sample I is taken in situ using the sample box, and the undisturbed water is taken from the river nearby the site;
B. the sample I is put in sample box to form sample, a lower porous stone 8 is disposed on the lower end of sample, an upper porous stone is disposed on the upper end of sample, filled with the undisturbed water for saturated immersion;
C. the circulating pump and/or heating unit is actuated simulate the flow of water body and the original ambient temperature of water body, the water flow velocity is controlled by controlling the circulating pump to be coincident with the water velocity of the original water body; afterwards, the jacking device is actuated to make the ejector rod props the upper porous stone downwards for strain controlled consolidation test, or the jacking device is turned off; the weights are applied to the circular tray to perform the stress controlled consolidation test;
D. the jacking device, circulating pump and heating unit are turned off, and the outer clamping plate of right side plate is removed according to the primal environment of sample I, the buffer bails wrapped in a net bag are disposed on the outer side of inner baffle of right side plate, and the circulating pump and/or heating unit is actuated again to simulate the flow of water body and the original ambient temperature of water body for a period of time, the mechanical data after the soil mass is washed are tested again, and the morphology of the sample II after washing is filmed;
E. the data from Step C and Step D are combined for analysis.
